(19) United States
(12) Patent Application Publication
(10) Pub. No.: US 2008/0138289 A1
(43) Pub. Date: Jun. 12, 2008

(54) SYSTEMS AND METHODS FOR DETECTING INFRARED EMITTING COMPOSITES AND MEDICAL APPLICATIONS THEREFOR

(75) Inventors: Jeffrey GORONKIN, Clifton Park, NY (US); Daniel LANDRY, Clifton Park, NY (US); Wei LIU, Schenectady, NY (US)

Correspondence Address: KENYON & KENYON LLP, 1500 K STREET N.W., SUITE 700, WASHINGTON, DC, 20005, US

(73) Assignee: EVIDENT TECHNOLOGIES, INC., Troy, NY (US)

(21) Appl. No.: 11/687,306

(22) Filed: Mar. 16, 2007

Related U.S. Application Data
(60) Provisional application No. 60/873,533, filed on Dec. 8, 2006.

(51) Int. Cl. A61K 49/04

(52) U.S. Cl. 424/9.4; 600/407; 604/264; 606/2; 623/1.13

(57) ABSTRACT

Medical applications for an infrared emitting composite are provided. The infrared emitting composite includes an infrared emitting agent dispersed in a matrix material, where the composite emits light of a wavelength range substantially non-absorbent to animal fluid or tissue. A system and method for detecting an infrared emitting composite are also provided. Exemplary applications for an infrared emitting composite include medical devices and pharmaceutical compositions.

### CROSS-REFERENCE TO RELATED APPLICATIONS

The present application claims priority to U.S. Provisional Application Ser. No. 60/873,533, filed Dec. 8, 2006, which is incorporated by reference herein.

### FIELD OF THE INVENTION

The present invention relates generally to medical applications for infrared emitting composites, such as medical devices and pharmaceutical compositions comprising infrared emitting composites. The present invention relates further to systems and methods for detecting infrared emitting composites.

### BACKGROUND OF THE INVENTION

In vivo medical procedures can be difficult to perform because the target body area may be obscured by body fluid or tissue, in close proximity to other vital body areas, and/or difficult to reach without highly invasive techniques. However, because in vivo procedures in general are becoming increasingly less invasive, they are preferable. The performance of in vivo procedures can be improved through the use of optical imaging to allow the physician to “see” the target area. To do so, optical imaging typically should be capable of imaging through body fluid and tissue well.

FIG. 1 shows the absorption spectrum for the human hand. This figure shows how strongly the hand's fluid and tissue absorb light at given wavelengths. Here, the hand's fluid and tissue are least absorbent of light having wavelengths from about 700 nm to about 800 nm. An additional area on the absorption spectrum wherein tissue and fluid in the hand are not strong absorbers exists in the wavelength range of about 1100 nm to about 1300 nm (not shown). Generally, the effective wavelength range for imaging through body fluid and tissue is from about 600 nm to about 1100 nm, preferably from about 650 nm to about 1000 nm, more preferably from about 700 to about 800 nm. Additionally, hemoglobin may be readily distinguished from surrounding tissue in the about 600 nm to about 1100 nm range.

Accordingly, in order for in vivo optical imaging to be effective, the optical imaging system should be capable of emitting and detecting light of the wavelength range from about 600 nm to about 1100 nm.

Some low-molecular weight organic dyes have been used in in vivo optical imaging, such as near-infrared (NIR) fluorescence imaging of the vasculature, to detect normal tissue, tumor vascular, bleeding, and/or tissue perfusion during surgery. These dyes have been typically administered to patients using a variety of injection procedures directed toward the target area. Upon excitation, the dyes have then emitted light in the NIR range to illuminate the target body area.

However, in some cases, these organic dyes may be easily photobleached, which may restrict their use to short-term imaging applications. They may have fairly low quantum yields (i.e., the percent of absorbed photons that are reemitted as photons), which reduces how visible they are during imaging. They may also be difficult to excite, often requiring a narrow wavelength excitation source, such as a laser, which may be either unavailable and/or expensive.

Current monitoring methods include known imaging techniques, such as x-ray imaging and magnetic resonance imaging (MRI), and indirect techniques, such as item inventory tracking and vital signs monitoring. However, these techniques are limited in their monitoring, primarily because of obscuring fluid and tissue, which are unavoidable in in vivo procedures.

Thus, there is a need in the art for better medical imaging technology.

### SUMMARY OF THE INVENTION

In an embodiment, the present invention provides a medical composition comprising a light emitting composite comprising a matrix material comprising a light emitting agent, where the light emitting composite emits light of a wavelength range that is substantially non-absorbent to animal fluid or tissue.

In another embodiment, the present invention provides a system comprising a medical composition comprising a light emitting composite, an excitation source capable of exciting the composite to emit light of a wavelength range that is substantially non-absorbent to animal fluid or tissue, and a detection device capable of detecting an emission from the excited composite.

In another embodiment, the present invention provides a method comprising providing a medical composition comprising a light emitting composite, exciting the composite to emit light of a wavelength range that is substantially non-absorbent to animal fluid or tissue, and detecting an emission from the excited composite.

### BRIEF DESCRIPTION OF THE DRAWINGS

FIG. 1 is a graph illustrating the absorption spectrum for the human hand.

FIG. 2 is a schematic illustration of an infrared emitting composite according to an embodiment of the present invention.

FIG. 3 is a schematic illustration of an infrared emitting composite according to another embodiment of the present invention.

FIGS. 4A-4D are schematic illustrations of semiconductor nanocrystal compositions that may be included in an infrared emitting composite according to embodiments of the present invention.

FIG. 5 is a flow chart of a method of making an infrared emitting composite according to an embodiment of the present invention.

FIG. 6 is a schematic illustration of a system for detecting an infrared emitting composite according to an embodiment of the present invention.

FIG. 7 is a method of detecting an infrared emitting composite according to an embodiment of the present invention.

FIG. 8 is an exemplary catheter that includes an infrared emitting composite according to an embodiment of the present invention.

FIG. 9 is an exemplary catheter that includes an infrared emitting composite according to another embodiment of the present invention.

FIG. 10 is an exemplary gauze pad that includes an infrared emitting composite according to another embodiment of the present invention.

FIG. 11 is an exemplary gauze pad that includes an infrared emitting composite according to another embodiment of the present invention.

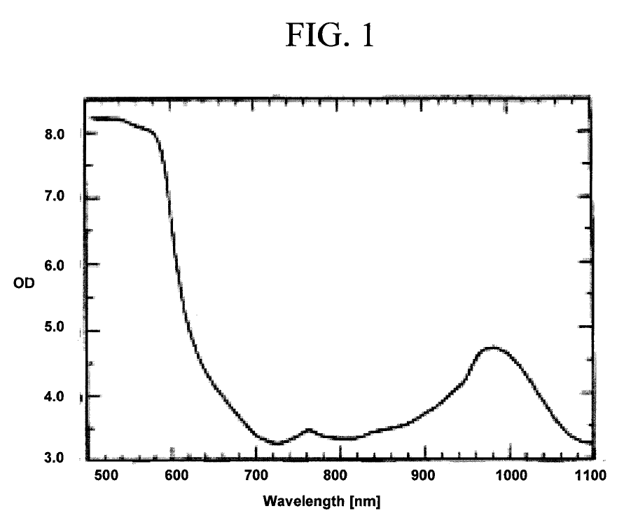

### DETAILED DESCRIPTION OF THE INVENTION

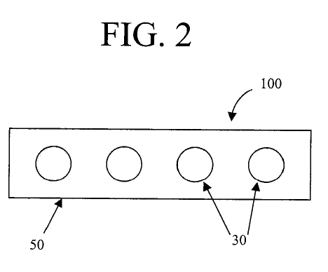

The present invention provides medical compositions suitable for in vivo use comprising light emitting compositions. For example, referring to FIG. 2, in an embodiment, the present invention provides an infrared emitting composite 100 comprising an infrared emitting agent 30 dispersed in a matrix material 50. In some embodiments, the infrared emitting agent 30 may be a fluorescent optical agent, such as visible emitting and NIR emitting organic fluorescent dyes. Non-limiting examples of visible emitting dyes that may be used include fluorescein. Non-limiting examples of NIR emitting dyes that may be used include cyanine dyes, such as Cy 5, Cy 5.5; derivations of indocyanine (ICG); carboxylic acid based dyes, such as the carboxylic acid of IRDye78 (IRDye78-CA); rhodamine B; diethylthiatricarbocyanine iodide (CTTCI); or suitable combinations thereof. Generally, indocyanine absorbs light having a wavelength at 780 nm and emits light having a wavelength at 830 nm with a quantum yield of 1.6%.

In some other embodiments, the infrared emitting agent 30 may be an inorganic phosphor, such as lanthanide based phosphors, including europium oxide and yttrium aluminum garnet phosphor. Generally, lanthanide-based phosphors emit light at wavelengths greater than 600 nm. Inorganic phosphors are typically used in light emitting diodes (LEDs) and display devices. These phosphors can easily be dispersed in a variety of matrix material.

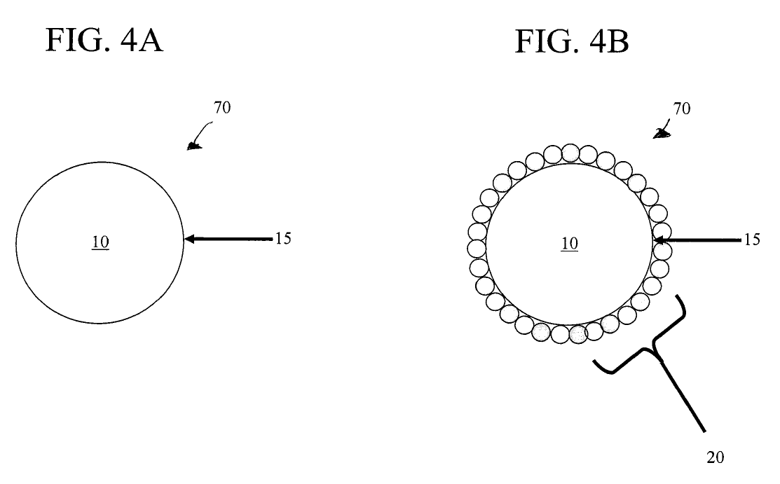

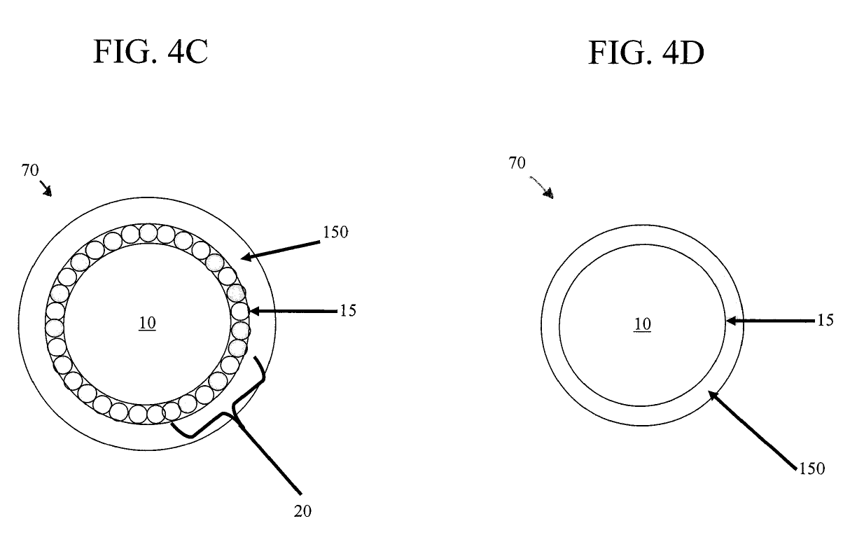

In other embodiments, the infrared emitting agent 30 may be a semiconductor nanocrystal composition. FIGS. 4A-4D are schematic illustrations of semiconductor nanocrystal compositions that may be used as the infrared emitting agent 30 in embodiments of the present invention.

Referring to FIG. 4A, in an embodiment, an infrared emitting agent comprises a semiconductor nanocrystal composition 70 comprising a semiconductor nanocrystal core 10 (also known as a semiconductor nanoparticle or semiconductor quantum dot) having an outer surface 15. Semiconductor nanocrystal core 10 may be spherical nanoscale crystalline materials (although oblate and oblique spheroids can be grown as well as rods and other shapes) having a diameter of less than the Bohr radius for a given material and typically but not exclusively comprises one or more semiconductor materials. Non-limiting examples of semiconductor materials that semiconductor nanocrystal core 10 can comprise include, but are not limited to, ZnS, ZnSe, ZnTe, CdS, CdSe, CdTe, HgS, HgSe, HgTe (II-VI materials), PbS, PbSe, PbTe (IV-VI materials), AlN, AlP, AlAs, AlSb, GaN, GaP, GaAs, GaSb, InN, InP, InAs, InSb (III-V materials), CuInGaS₂, CuInGASe₂, AgInS₂, AgInSe₂, AuGaTe₂ (I-III-VI materials), or suitable combinations thereof. In addition to binary and ternary semiconductors, semiconductor nanocrystal core 10 may comprise quaternary or quintary semiconductor materials. Non-limiting examples of quaternary or quintary semiconductor materials include AₓB_{y}C_{z}D_{w}E₂ᵥ wherein A and/or B may comprise a group I and/or VII element, and C and D may comprise a group III, II and/or V element although C and D cannot both be group V elements, and E may comprise a VI element, and x, y, z, w, and v are molar fractions between 0 and 1.

Referring to FIG. 4B, in an alternate embodiment, one or more metals 20 are formed on outer surface 15 of semiconductor nanocrystal core 10 (referred to herein as “metal layer” 20) after formation of core 10 to form the nanocrystal composition 70. Metal layer 20 may act to passivate outer surface 15 of semiconductor nanocrystal core 10 and limit the diffusion rate of oxygen molecules to semiconductor nanocrystal core 10. According to the present invention, metal layer 20 is formed on outer surface 15 after synthesis of semiconductor nanocrystal core 10 (as opposed to being formed on outer surface 15 concurrently during synthesis of semiconductor nanocrystal core 10). Metal layer 20 is typically between 0.1 nm and 5 nm thick. Metal layer 20 may include any number, type, combination, and arrangement of metals. For example, metal layer 20 may be simply a monolayer of metals formed on outer surface 15 or multiple layers of metals formed on outer surface 15. Metal layer 20 may also include different types of metals arranged, for example, in alternating fashion. Further, metal layer 20 may encapsulate semiconductor nanocrystal core 10 as shown in FIG. 4B or may be formed on only parts of outer surface 15 of semiconductor nanocrystal core 10. Metal layer 20 may include the metal from which the semiconductor nanocrystal core is made either alone or in addition to another metal. Non-limiting examples of metals that may be used as part of metal layer 20 include Cd, Zn, Hg, Pb, Al, Ga, In, or suitable combinations thereof.

Semiconductor nanocrystal core 10 and metal layer 20 may be grown by the pyrolysis of organometallic precursors in a chelating ligand solution or by an exchange reaction using the prerequisite salts in a chelating ligand solution. The chelating ligands are typically lyophilic and have an affinity moiety for the metal layer and another moiety with an affinity toward the solvent, which is usually hydrophobic. Typical examples of chelating ligands include lyophilic surfactant molecules such as Trioctylphosphine oxide (TOPO), Trioctylphosphine (TOP), Tributylphosphine (TBP), Hexadecyl amine (HDA), Dodecanethiol, and Tetradecyl phosphonic acid (TDPA), or suitable combinations thereof.

Referring to FIG. 4C, in an alternate embodiment, an infrared emitting agent comprises a nanocrystal composition 70 further comprising a shell 150 overcoating metal layer 20. Shell 150 may comprise a semiconductor material having a bulk bandgap greater than that of semiconductor nanocrystal core 10. In such an embodiment, metal layer 20 may act to passivate outer surface 15 of semiconductor nanocrystal core 10 as well as to prevent or decrease lattice mismatch between semiconductor nanocrystal core 10 and shell 150.

Shell 150 may be grown around metal layer 20 and is typically between 0.1 nm and 10 nm thick. Shell 150 may provide for a type A semiconductor nanocrystal composition 70. Shell 150 may comprise various different semiconductor materials such as, for example, CdSe, CdS, CdTe, ZnS, ZnSe, ZnTe, HgS, HgSe, HgTe, InP, InAs, InSb, InN, GaN, GaP, GaAs, GaSb, PbSe, PbS, PbTe, CuInGaS₂, CuInGaSe₂, AgInS₂, AgInSe₂, AuGaTe₂, ZnCuInS₂ or suitable combinations thereof.

Semiconductor nanocrystal core 10, metal layer 20, and shell 150 may be grown by the pyrolysis of organometallic precursors in a chelating ligand solution or by an exchange reaction using the prerequisite salts in a chelating ligand solution. The chelating ligands are typically lyophilic and have an affinity moiety for the shell and another moiety with an affinity toward the solvent, which is usually hydrophobic. Typical examples of chelating ligands 160 include lyophilic surfactant molecules such as Trioctylphosphine oxide (TOPO), Trioctylphosphine (TOP), Tributylphosphine (TBP), Hexadecyl amine (HDA), Dodecanethiol, Tetradecyl phosphonic acid (TDPA), or suitable combinations thereof.

Referring to FIG. 4D, in an alternate embodiment, the present invention provides a nanocrystal composition 70 comprising a semiconductor nanocrystal core 10 having an outer surface 15, as described above, and a shell 150, as described above, formed on the outer surface 15 of the core 10. The shell 150 may encapsulate semiconductor nanocrystal core 10 as shown in FIG. 4D or may be formed on only parts of outer surface 15 of semiconductor nanocrystal core 10.

A semiconductor nanocrystal composition used as an infrared emitting agent is electronically and chemically stable with a high luminescent quantum yield. Chemical stability refers to the ability of a semiconductor nanocrystal composition to resist fluorescence quenching over time in aqueous and ambient conditions. Preferably, the semiconductor nanocrystal compositions resist fluorescence quenching for at least a week, more preferably for at least a month, even more preferably for at least six months, and even more preferably for at least a year, including all intermediate values therebetween. Electronic stability refers to whether the addition of electron or hole withdrawing ligands substantially quenches the fluorescence of the semiconductor nanocrystal composition. Preferably, a semiconductor nanocrystal composition would also be colloidally stable in that when suspended in organic or aqueous media (depending on the ligands) they remain soluble over time. Preferably, a high luminescent quantum yield refers to a quantum yield of at least 10%. Quantum yield may be measured by comparison to Rhodamine 6G dye with a 488 excitation source. Preferably, the quantum yield of the semiconductor nanocrystal composition is at least 25%, more preferably at least 30%, still more preferably at least 45%, and even more preferably at least 55%, and even more preferably at least 60%, including all intermediate values therebetween, as measured under ambient conditions.

A semiconductor nanocrystal composition 70 can produce strong emissions in the NIR when the bandedge emission of the underlying core 10 is at higher energy than the wavelength range of interest.

An infrared emitting agent of the present invention is prepared so that it may be imaged through animal tissue. As discussed above, most tissue is only relatively transparent to light at wavelengths between about 600 nm and about 1100 nm. Therefore, any fluorescing agent that emits light in these wavelengths can not be strongly absorbed by the animal's fluid, e.g., hemoglobin, and tissue. Additionally, the exact emission wavelength of an infrared emitting agent of the present invention may depend on such factors as the amount of tissue or hemoglobin between the agent and the detection device and the sensitivity of the detection device itself.

Referring again to FIG. 2, an infrared emitting agent 30, according to certain embodiments of the present invention, is dispersed in a matrix material 50. The matrix material 50 may be any material capable of including the infrared emitting agent 30 and that does not absorb or otherwise interfere with the emissions from the agent 30. Non-limiting examples of matrix material 50 that may be used include glass, plastic, metals and other polymers such as acetal, ethylene tetrafluoroethylene (ETFE), ethylene vinyl acetate (EVA), fluorinated ethylene propylene (FEP), high density polyethylene (HDPE), low density polyethylene (LDPE), nylon (6, 11, 12), perfluoroalkoxy (PFA), polycarbonate (PC), polyether block amide (PEBA), polypropylene (PP), polytetraflouroethylene (PTFE), aliphatic polyurethane (PUR), and aromatic polyurethane (PUR), polyvinyl chloride (PVC), polyvinyl alcohol (PCVA), polyacrylic acid, polymethyl methacrylate, and any combinations thereof. The agent 30 may be dispersed in the matrix material 50 using any known techniques. For example, if an infrared emitting agent is a semiconductor nanocrystal, siloxane-containing ligands on the surfaces of the nanocrystals can be crosslinked with tellurium oxide in a glass matrix material, via a condensation reaction, to produce a composite 100 containing semiconductor nanocrystals and silicon oxide. Therefore, in certain embodiments, the compositions are silicone-based composites that can be used in silicone-based medical devices. The agent 30 may be dissolved, suspended, reacted, or otherwise dispersed in the matrix material 50, thereby forming an infrared emitting composite 100.

An infrared emitting agent/polymer matrix composite 100 may be formed into a medical composition adapted for in vivo use. A medical composition can be any type of medical composition adapted for in vivo use, such as, for example, a medical device, medical instrument, coating, or pharmaceutical composition. In embodiments where the medical composition is a device or apparatus, the device or apparatus can be formed by well-known molding techniques such as injection and extrusion molding. In certain embodiments, a medical composition is an insertable medical device such as, for example, a catheter or a guidewire. In certain embodiments, the insertable medical device is an implantable medical device such as, for example, a shunt, a filter, a graft, a lead, a scaffold, a plug, a stent, a mechanical heart valve, or other types of implants. In certain embodiments, the implantable medical device is a minimally invasive medical device. The medical composition may also be an absorbent medical substrate, such as, for example, a gauze pad, a sponge or a bandage. In other embodiments, the medical composition is a medical instrument such as, for example, an endoscope, a scalpel, a clamp, etc.

In embodiments where the matrix material is a glass, an infrared emitting agent/glass matrix composite 100 may be easily fashioned into any shape or form, including an optical fiber, using molding and extrusion techniques. Additionally or alternatively, a medical composition may be a coating on a medical device or medical instrument. Additionally or alternatively, a composite 100 may be injected into a hollow portion of a medical device and/or instrument. In other embodiments, a medical composition is a pharmaceutical composition. The pharmaceutical composition may be in solid, semi-solid, liquid or gas form. Non-limiting examples of pharmaceutical compositions include an ointment, a cream, a gel, a pill, or a vapor. The pill may be a capsule or tablet.

As mentioned above, a non-limiting example of an infrared emitting agent/polymer matrix composite is a semiconductor nanocrystal/silicone composite. Previously, semiconductor nanocrystals could not be dispersed in silicone because the platinum catalyst used to polymerize the silicone could poison the nanocrystals. However, in relevant embodiments of the present invention, this problem is eliminated, thereby making it possible to provide semiconductor nanocrystal/silicone composites.

In certain embodiments, a composite is an ink, paint, or dye. An infrared emitting agent/ink or paint or dye matrix composite 100 may be used to print, e.g., a bar code, directly onto a medical device or instrument, into or onto another material, such as an adhesive, or onto a label to be affixed to a medical device or instrument. The printed composite 100 may optionally be coated with an impermeable material. The composite 100 may be printed by any known technique, such as, but not limited to, inkjet printing, thermal transfer printing, thermal direct printing, flexographic printing, heatset printing, screen printing, gravure printing, lithographic printing, etc.

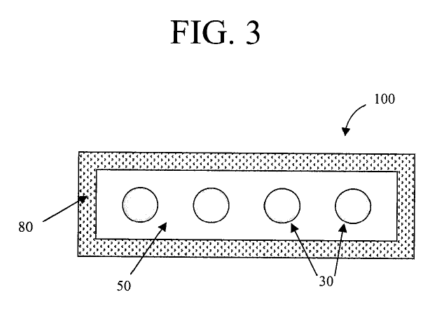

Referring to FIG. 3, in an alternate embodiment, to minimize contact with an animal's body, the composite 100 may further comprise a biocompatible material 80 coating the matrix material 50. Although not limited to any particular use, such an embodiment may be used in embodiments where matrix material 50 is not biocompatible. The biocompatible material 80 may encapsulate the matrix material 50 as shown in FIG. 3 or may coat certain portions of the material 50 that are likely to contact the animal's body. The biocompatible material 80 may be impermeable, semi-impermeable, or permeable, depending on the application in which the composite 100 is used. The matrix material 50, the biocompatible material 80, or both may sufficiently seal the infrared emitting agent 30 therein to reduce any risk of toxicity to the body.

In other embodiments, the infrared emitting composite 100 may comprise other contrast agents along with the infrared emitting agent 30 in order to provide a dually functional composite structure that can be imaged by one or more imaging techniques. For example, other contrast agents that may be used include tracers detectable by x-ray imaging, positron emission tomography (PET), and magnetic resonance imaging (MRI).

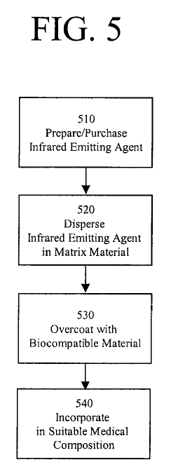

FIG. 5 is a flow chart of an exemplary method for making an infrared emitting composite according to an embodiment of the present invention. In step 510, the infrared emitting agent may be prepared or purchased. The preparation of the agent may be according to known techniques for that agent. In step 520, the agent may be dispersed into a matrix material to form a composite. The matrix material may be selected based on its compatibility with the agent and its absorption characteristics. The dispersing of the agent into the matrix material may be according to known techniques for that agent and matrix material. In step 530, optionally, the matrix material with the dispersed agent may be overcoated with a biocompatible material to form a composite. The biocompatible material may be selected based on its compatibility with the agent and matrix material, as well as the animal with which the composite will make contact, and the biocompatible material's absorption characteristics. The overcoating may be according to known techniques for that biocompatible material. In step 540, the composite may be incorporated into a medical composition such as a medical device, a pharmaceutical composition, or any other suitable medical application. The incorporation may involve molding or forming the composite into a shape suitable for the particular application; disposing the composite into or onto a device, composition, or any other suitable medical application; embedding the composite into a device, composition, or any other suitable medical application; or any other incorporation technique.

For example, dry semiconductor nanocrystals can be compounded into silicone materials such as liquid silicone rubber (LSR) materials. Then materials can then be extruded directly, co-extruded with gum rubber silicone, or molded into silicone parts. Alternatively or in addition, a thin thread can be coated with IR luminescent paint and the thread can be co-extruded with the silicone gum rubber or polymer, thereby becoming encased. The thread can be fabricated from a polymer or a natural fiber. The thread can also be overcoated with a polymer creating a barrier between the light emitting agent and the biological environment. In certain embodiments, the thread is co-extruded into the lumen of catheter, molded into a medical device, or applied to the surface of a coated medical device (including being sealed within such polymer by applying another layer of polymer on the thread). In certain embodiments, the thread has a predetermined pattern. In certain embodiments, the thread has a diameter of approximately 0.01 inches. Still alternatively or in addition, dry semiconductor nanocrystals can be compounded into a silicone coating material and the material can be used to dip coat medical instruments for example.

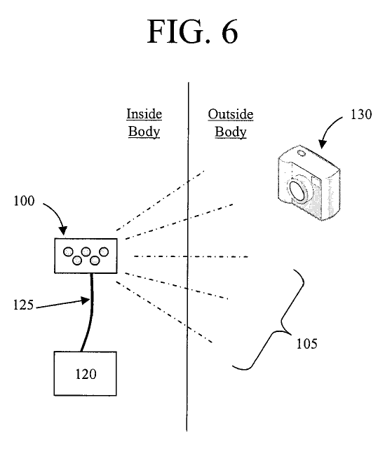

Referring to FIG. 6, in an embodiment, the present invention provides a system for detecting an infrared emitting composite. The system comprises an infrared emitting composite 100 incorporated into a medical composition, an excitation source 120, and an emission detection device 130.

The infrared emitting composite 100, as described above, may emit light 105 at wavelengths from about 600 nm to about 1100 nm. The infrared emitting composite 100 may be inside the animal's body, as shown in FIG. 6. Alternately, the composite 100 may be on the animal's skin or proximate thereto.

The excitation source 120 is a source capable of exciting the composite 100 so that the composite 100 will emit light at the desired wavelengths. Non-limiting examples of an excitation source that may be used include a white light source, a light emitting diode, a laser, a chemiluminescent material, and any other source capable of exciting the composite. Here, the excitation source transmits an optical signal to the composite 100 to excite the composite 100. The optical signal is transmitted by communication medium 125. The communication medium 125 may be a fiber optic cable, a wireless transmitter/receiver, etc. capable of transmitting an optical signal. The excitation source 120 may be inside an animal's body with the composite 100, as shown in FIG. 6. Alternately, the excitation source 120 may be on the animal's skin while in communication with the composite 100 inside the body or on the skin. Or, the excitation source 120 may be at a position away from the body while in communication with the composite inside the body or on the skin.

The emission detection device 130 is a device capable of detecting an emission from the composite 100. Non-limiting examples of an emission detection device that may be used include a CCD camera, a night vision scope, and any other device capable of detecting at the wavelengths of the emission from the composite 100. The device 130 may be tuned or tunable to the wavelengths of the emission. The device 130 may then detect the light 105 emitted from the excited composite 100 at those wavelengths. The device 130 may be at a position away form the body, as shown in FIG. 6, while in communication with the composite 100 inside the body or on the skin. Alternatively, the device 130 may be on the skin while in communication with the composition 100 inside the body or on the skin. Or, the device 130 may be inside the body with the composite 100. The device 130 may include a filter to adequately filter out light from the excitation source 120, thereby increasing the signal to noise ratio of the emission from the composite 100 over what it would be if the excitation source's light interferes.

It is to be appreciated that a system for detecting an infrared emitting composite is not limited to that illustrated in FIG. 6. Rather, the system may include other and/or additional components in other types, forms, and configurations.

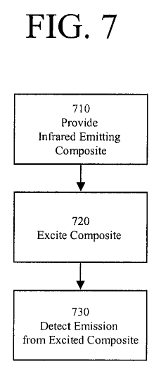

FIG. 7 is a method for detecting an infrared emitting composite according to an embodiment of the present invention. In step 710, an infrared emitting composite is provided. The composite may be provided in a medical composition. For example, the composite may be incorporated into fiber optics and thin polymer strands, plastic or composite sheets, pills, capsules, or other compositions, glass, and a wide variety of molded, formed, coated, embedded and/or otherwise fabricated medical devices that enable surgeons to, for example: visualize and/or monitor the location of the device or composition incorporating the infrared emitting composite or the body components in contact with the composite; create optical maps; and avoid lacerations and other damage to critical body components. Known incorporation methods may be used. The medical application may be therapeutic or diagnostic.

In step 720, an excitation source excites the composite. Several different techniques can be employed to excite the composite of the present invention. The type of excitation used varies greatly with the application. Non-limiting examples of how the composite may be excited are as follows.

In some embodiments, the composite is excited using broad spectrum white light. In certain embodiments, the white light is first filtered to remove the NIR component in order to increase the signal to noise ratio of the white light signal. In other embodiments, filtering is not necessary as long as the NIR component of the light is significantly weaker than the visible component of the light and the emission detection device is tuned or filtered for infrared detection. The white light may then be optically transmitted to the composite. Alternately, the white light may be directly coupled to the composite through a optical fiber, for example, without going through tissue.

In some other embodiments, the composite is excited using a laser. The laser may either directly excite the composite or excite the composite through the body's tissue. To excite the composite through the body's tissue, the laser will transmit an excitation laser beam of appropriate wavelength through the tissue to reach the composite. To excite the composite directly, the laser is coupled directly to the composite via an optical fiber, for example, and transmits the laser beam through the fiber to the composite. Alternatively, the laser is within close proximity to the composite and transmits the laser beam across the short distance to the composite without going through tissue.

In some other embodiments, the composite is excited using a light emitting diode (LED). The LED may either directly excite the composite or excite the composite through the body's tissue. Additionally, the LED and its power source may be completely contained with the composite. For example, an ingestible capsule can contain a blue LED with an internal power source. The composite material forming the capsule may contain an infrared emitting agent, such as PbS semiconductor nanocrystal compositions, and will emit light at the desired wavelengths when the LED inside the capsule is activated.

In some other embodiments, the composite is excited using tags, e.g., radio-frequency identification (RFID) tags. A tag may activate the light source that provides the optical signal to excite the composite. The tag may activate the light source directly or through the body's tissue. The light source may then excite the composite directly or through the body's tissue, depending on its position. For example, the tag may be attached to a light source, for example an LED, and may be used as a switch to activate the LED, which then excites the composite, thereby providing emissions on demand.

In some other embodiments, the composite is excited by chemiluminescence. Chemiluminescence is the emission of light without emission of heat as the result of a chemical reaction. In certain embodiments, chemiluminescent material is included with the infrared emitting agent in the composite. The chemiluminescent material reacts and emits light to excite the infrared emitting agent in the composite, without the need for an external source. Chemiluminescence may excite the composite for long periods of time without external stimulus, particularly where other excitation sources may not be practical.

In certain other embodiments, the composite is excited by a two-photon absorption. Two-photon absorption is a technique in which the infrared emitting agent absorbs two infrared photons simultaneously, which means that the agent absorbs enough energy to be raised into the excited state. The photons may be transmitted to the composite by any capable excitation source, such as, for example, a laser. The agent then emits a single photon with a wavelength that is characteristic of the agent material. Because two photons are absorbed to excite the agent, the probability that the agent will then emit a single photon is related to the intensity, squared, of the excitation source. As such, the excited agent is most likely to emit within the focal volume of the excitation beam.

Referring again to FIG. 7, after the composite is excited and emits light at the desired wavelengths, in step 730, the emission detection device detects the emission.

Non-limiting examples of medical applications for infrared emitting composites are as follows.

### EXAMPLES

### Example 1

### Incorporating PbS Dots into Silicone Rubber

To a 12 milliliters (ml) centrifuge tube, 0.1 ml PbS dot (˜1 g/ml) was added. The dots were precipitated down by adding 4 ml methanol. After spinning at 400 rpm for 4 minutes, the supernatant was removed, and 0.1 ml chloroform was added to re-dissolve the dots. 10 grams liquid silicone material was made by mixing equal amount of Dow Corning LSR Q7-4850 part A and part B together in a 50 ml beaker. The washed PbS dots were added into the beaker, and thoroughly mixed with the silicone material. The mixture of the PbS dots and silicone was degassed under vacuum for 10 minutes.

### Example 2

### Stents with Infrared Emitting Agents

A stent may incorporate an infrared emitting composite of the present invention. A stent is a medical device used to overcome decreases in vessel or duct diameter in the body. A stent is often used to reduce pressure differences in blood flow to organs caused by an obstruction, in order to maintain an adequate delivery of oxygen to the organs. A stent is most popularly used for coronary arteries, but may be used for other body areas, such as peripheral arteries and veins, bile ducts, esophagus, colon, trachea or large bronchi, ureters, and urethra.

The infrared emitting composite may be incorporated in the stent in various ways. The composite may be disposed as a polymer coating on a catheter having the stent on its distal end or as a polymer coating on the stent itself. Alternatively, the composite may be placed within reservoirs of an uncoated stent. Alternatively or in addition, the composite as a polymer may fill a hollow section of the stent catheter. Alternatively or in addition, the composite may be incorporated directly in the material comprising the stent or catheter. Alternatively or in addition, the composite may be incorporated into the material of a fiber optic bundle placed within the stent catheter. Other methods of incorporating an infrared emitting composite in a stent are also possible and the aforementioned methods are simply exemplary.

In an embodiment, the infrared emitting agent of the composite may be tuned to emit light at wavelengths that are substantially non-absorbent to blood and tissue. As the stent is positioned in a body, the infrared emitting agent may be excited using, for example, a strong white light source that is filtered to only allow the visible emitting spectra to pass; a high power laser; a fiber optic internalized in the stent catheter; or through a chemiluminescent material inside the catheter. The detection device may be positioned over the body or in the body, depending on the configuration of the detection system, to assist a surgeon in determining the position of the stent. The detection device results may be combined with alternate detection results, such as from optical tomography, to detect the stent in less transparent body areas, such as areas of high plaque concentration.

In certain embodiments, multiple infrared emitting composites may be needed. As such, each composite may be tuned to emit light at a uniquely identifiable wavelength within the appropriate range. Multiple excitation sources may be used that are capable of exciting the composites at the different wavelengths or a single source capable of excitation and multiple wavelengths may be used. The detection device may be tuned to detect emissions over the appropriate range or multiple detection devices may be used. As such, each stent catheter or any other medical device may incorporate the unique composite and be differentiated from every other catheter by the unique emission wavelength.

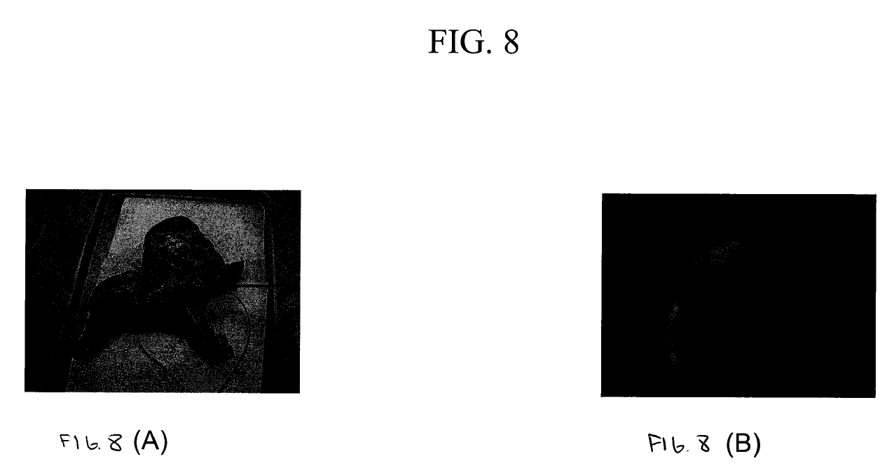

FIG. 8 is an example of a stent catheter having an infrared emitting composite according to an embodiment of the present invention. In this example, the stent catheter was devised by injecting 2% loaded 850 nm emitting semiconductor nanocrystal polymer emulsions, prepared using known techniques, comprising the infrared emitting composite into a hollow space in a narrow polyethylene tube comprising the stent catheter. The tube was filled to approximately 18 inches and tied at both ends.

Two excitation sources were used to excite the infrared emitting composite in the tube: a 1 mW red 660 nm laser and the room lighting of white lights. The laser was effective for illumination in deep tissue, at a depth of greater than 1 cm.

A near-infrared sensitive detector with an 800 nm long pass filter was used to detect the emissions from the infrared emitting composite.

As shown in FIG. 8, the stent catheter was inserted into a swine heart artery and the tissue surrounding the catheter illuminated by the laser and room lighting. See FIG. 8A. The emission from the composite in the catheter is clearly visible through the swine tissue as detected by a camera through a photomultiplier night vision device. See FIG. 8B.

A stent catheter having an infrared emitting composite can be used to visualize vasculature and stent placement in both open cavity and laparoscopic procedures.

### Example 3

### Catheters with Infrared Emitting Agents

As described above with the stent catheter, other catheters may incorporate an infrared emitting composite according to an embodiment of the present invention. The catheter is a medical device that may be inserted into the body for various surgical and diagnostic purposes. The composite may be incorporated in the catheter and detected, as described above for the stent catheter.

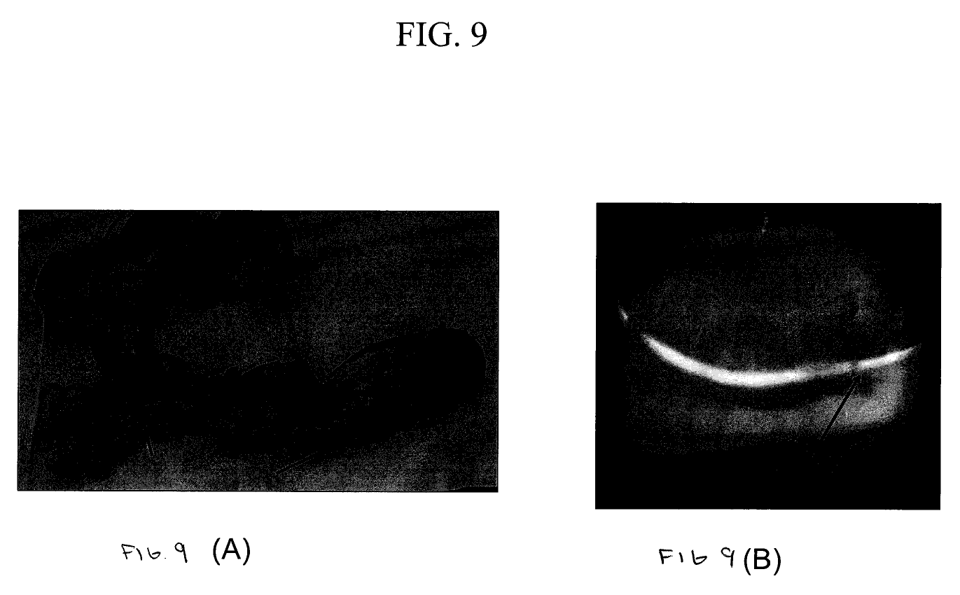

FIG. 9 is an example of a catheter having an infrared emitting composite according to an embodiment of the present invention. In this example, the catheter was devised by injecting 2% loaded 850 nm emitting semiconductor nanocrystal polymer emulsions, prepared using known techniques, comprising the infrared emitting composite into a hollow space in a narrow polyethylene tube comprising the stent catheter. The tube was filled to approximately 18 inches and tied at both ends.

Two excitation sources were used to excite the infrared emitting composite in the tube: a 1 mW red 660 nm laser and the room lighting of white lights. The room lighting was effective for thin tissue. The laser was effective for illumination in deep tissue, at a depth of greater than 1 cm.

A near-infrared sensitive detector with an 800 nm long pass filter was used to detect the emissions from the infrared emitting composite.

As shown in FIG. 9, the catheter was inserted into a swine ureter outside the bladder and passed through the kidney and the tissue surrounding the catheter illuminated by the laser and room lighting. See FIG. 9A The emission from the composite in the catheter is clearly visible through the swine ureter as detected by a camera through a photomultiplier night vision device. See FIG. 9B.

Although there are many uses of catheter having an infrared emitting composite, one particular use is for ureter marking. Over one million laparoscopic hysterectomies are preformed each year. One of the risks to the patient during this surgery is an inadvertent nick or laceration of the ureter. This is because the positioning of and connective tissue surrounding the ureter is very difficult to detect during surgery. Additionally, if damage to the ureter has inadvertently occurred, such damage is difficult to detect. Therefore, by threading a thin catheter comprising an infrared emitting composite into the ureter during the surgical procedure, the ureter can be clearly visualized through a laparoscopic scope fitted with an excitation source and an infrared sensitive camera. Software can be used to superimpose the detected emission over a visible image, giving the surgeon a clear picture of the ureter position and morphology. In addition, differences in the emission can be detected to determine whether or not the ureter has been accidentally damaged. As an alternative to the laparoscopic camera, a handheld detector using night vision technology or similar detectors may be used to detect the composite emission.

### Example 4

### Implants with Infrared Emitting Agents

An implant may incorporate an infrared emitting composite according to an embodiment of the present invention. The infrared emitting composite may be incorporated in the implant in various ways. The composite may be incorporated into a lining of the implant. Or the composite may be disposed as a polymer coating on the implant. Or the composite as a polymer may be added to the material filling the implant. Or the composite may be incorporated directly in the material comprising the implant. Other ways are also possible. The composite may be detected, as described above in the previous Examples.

Although the implants having infrared emitting agents have many different uses, implant diagnostics is a desirable application for an implant having an infrared emitting composite of the present invention. For example, a breast implant having an infrared emitting composite may be used to identify breast tissue density by measuring the emission from the composite. Current state of the art mammogram techniques require compression of the breast for proper imaging and can be painful to patients, particularly those with capsular contracture. For women who have breast implants, there is a possibility of rupturing the implant during the mammogram procedure. Additionally, because mammograms require the use of radiation, more images are often taken when implants are present because the implants tend to obscure the area that is being imaged. An infrared emitting breast implant provides a non-radioactive diagnostic alternative and may not require compression of the breast during imaging. Generally, injuries and tumors scatter light more strongly than healthy tissue because of the differences in vascular density. Therefore, the absorption characteristics of the tissues may be determined from the composite emission variations at different body locations. Hence, such anomalies as benign and malignant lesions, hemorrhages, and infection may be determined. Anomalies associated with the implant itself and surrounding tissue may also be determined. Alternate imaging techniques, such as diffuse optical tomography (DOT), computer tomography (CT), or magnetic resonance imaging (MRI), may be used in conjunction with the composite implant diagnostics.

Implant positioning is another desirable application for an implant having an infrared emitting composite of the present invention. For example, an infrared emitting composite incorporated in an implant may be used to identify the location of the implant during surgical placement to ensure that the implant is placed correctly in a body. Additionally, the composite may be used to identify any anomalies in surrounding tissue during and after implantation.

### Example 5

### Gauze and Sponges with Infrared Emitting Agents

Gauze and sponges may incorporate an infrared emitting composite according to an embodiment of the present invention. Gauze and sponges are often used during surgery to wipe up any blood or other fluids obscuring the surgical area. As they become fluid filled, they may be difficult to discern from body tissue. As such, they may be inadvertently left in the body after surgery, resulting in a second surgery to remove them and, in some cases, to also repair injury caused by them. It has been reported that one in ten thousand surgeries result in foreign objects left behind at the end of surgery. The current procedure is to count the gauze and sponges, etc., before surgery and then after surgery before closing the body. Sometimes, for high-risk patients, such as those involved in emergencies or lengthy surgeries, the patient is x-rayed for foreign objects before leaving the operating room. Therefore, an infrared emitting composite incorporated into the gauze and sponges may be used to track them during surgery so that they may be successfully retrieved at the end of surgery.

The infrared emitting composite may be incorporated in gauze and sponges in various ways. For example, the composite may be attached to the gauze and sponges or the composite may be woven into the gauze and sponges. Other ways are also possible.

The composite may be detected, as described above in previous Examples. Additionally, an audible sensor may be incorporated into the detector to emit an audible signal when the detector detects a composite emission from the gauze and sponges.

Each piece of gauze or sponge may be incorporated with a composite tuned to a uniquely identifiable emission wavelength within the infrared range. As such, the detector may detect multiple emissions at multiple wavelengths, thereby allowing the surgeon to differentiate between the gauze and sponges.

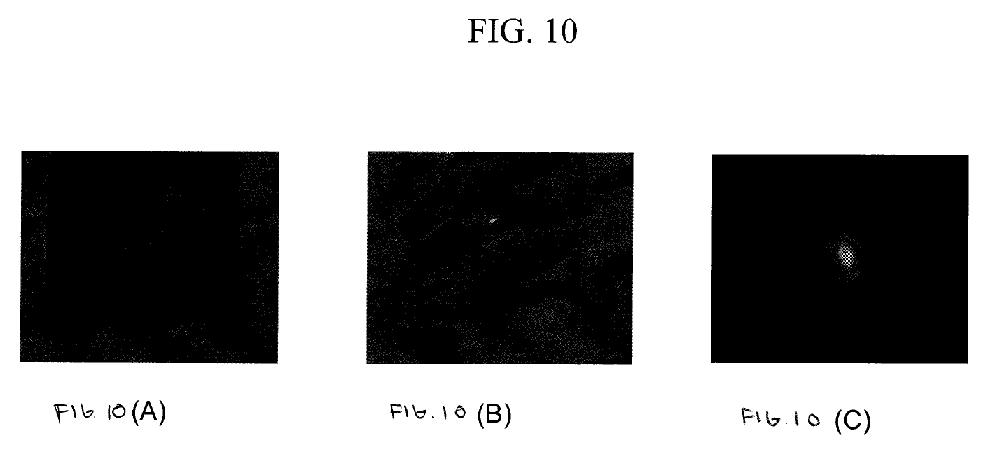

FIG. 10 is an example of a gauze pad having an infrared emitting composite according to an embodiment of the present invention. In this example, a 4-inch by 4-inch piece of gauze was incorporated with PbS semiconductor nanocrystal emitting agents in a polymer material comprising an infrared emitting composite. See FIG. 10A. The gauze was saturated with swine blood and then placed underneath a piece of swine skin 1.5 cm thick. The composite in the gauze was excited with a 1 mW read nm laser and the room lighting of white lights. See FIG. 10B. The emission from the composite in the gauze is clearly visible through the swine skin as detected by a hand held detector. See FIG. 10C.

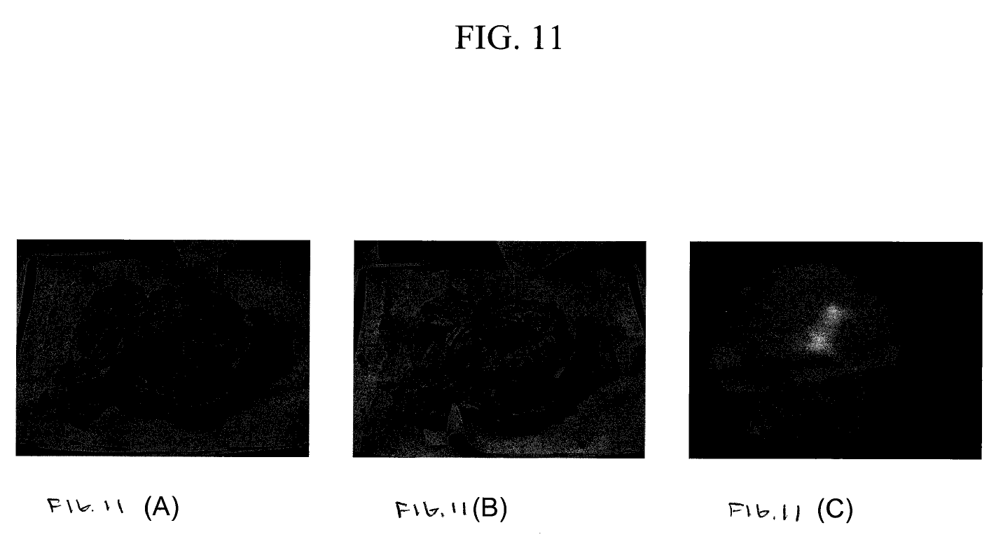

FIG. 11 is another example of the gauze pad of FIG. 10. In this example, the gauze was saturated with swine blood and then placed among swine organ remains. See FIG. 11A. A swine tissue lining was pulled over the gauze. See FIG. 11B. The emission from the composite in the gauze, while not visible to the naked eye, is clearly detected through the tissue by a hand held detector. See FIG. 11C.

In some embodiments, radio frequency identification (RFID) tags may be used in conjunction with infrared emitting composites of the present invention. RFID is a technique that uses tags or transponders attached to or implanted in objects to store and retrieve identification data about that object via radio waves. Gauze and sponges having infrared emitting composites may also incorporate RFID tags. The RFID tags may be used for inventory control and data storage/retrieval before, during, and after surgery. Since each RFID tag uses a different radio frequency, the gauze pad and sponges may be differentiated from each other by having RFID tags with unique identifiable radio frequencies. As such, when the detector detects the emissions from the composites in the gauze and sponges, the RFID frequencies may be coupled with the detection results to differentiate between the gauze and sponges.

Similar materials, such as bandages, wadding, adhesive tape, etc., may incorporate an infrared emitting composite according to an embodiment of the present invention.

### Example 6

### Surgical Instruments with Infrared Emitting Agents

A surgical instrument may incorporate an infrared emitting composite according to an embodiment of the present invention. Surgical instruments, such as a scalpel, a clamp, etc., may be obscured by blood or other fluids and tissue during surgery. As such, they may be inadvertently left in a body at the end of surgery. An infrared emitting composite incorporated in the instrument may be used to track the instrument so that it may be successfully retrieved at the end of surgery.

An infrared emitting composite may be incorporated in the surgical instrument in various ways. For example, the composite may be molded or fabricated into the instrument itself, the composite may be disposed as a polymer coating on the instrument, the composite as a polymer may fill a hollow in the instrument, or the composite may be printed on a label affixed to the instrument. Other ways are also possible. The composite may be detected, as described above in previous Examples.

In some embodiments, RFID tags may be used in conjunction with infrared emitting composites as described above regarding gauze and sponges.

### Example 7

### Image-Guided Devices with Infrared Emitting Agents

An image-guided device may incorporate an infrared emitting composite according to an embodiment of the present invention. An image-guided device is a medical device that the surgeon indirectly sees, e.g., via imaging, while in use. This device is typically used in minimally invasive surgeries, where the device is inserted into the body through a small incision or opening, moved to the surgical area, and then manipulated to perform the surgery at that area. Since the device is inside the body, the surgeon can not see the device and must rely on imaging to monitor the location of the body relative to the image-guided device. Typical imaging is done by fiber optic guides, internal video cameras, flexible or rigid endoscopes, ultrasonography, etc. Generally, multi-modal monitoring is used, taking data from such imaging sources as magnetic resonance imaging (MRI), fluoroscopy, computer tomography (CT), etc., to provide a three-dimensional view of the body during surgery.

The infrared emitting composite may be incorporated in the image-guided device in various ways. For example, the composite may be incorporated as described above in previous Examples. The composite may also be incorporated in an adhesive or cream to be applied to the skin to identify the body's location relative to the image-guided device.

### Example 8

### Capsules and Pills with Infrared Emitting Agents

A capsule or pill may incorporate an infrared emitting composite according to an embodiment of the present invention. The composite may be incorporated in the capsule or pill in various ways. For example, the composite may be disposed as a coating on the capsule or pill, the composite may form the capsule container and/or fill the container, or the composite may form the pill itself. Other ways are also possible.

After ingestion, the capsule or pill may be detected, as described above in previous Examples.

The foregoing description and example have been set forth merely to illustrate the invention and are not intended as being limiting. Each of the disclosed aspects and embodiments of the present invention may be considered individually or in combination with other aspects, embodiments, and variations of the invention. In addition, unless otherwise specified, none of the steps of the methods of the present invention are confined to any particular order of performance. Furthermore, any advantages described herein should not be read as limitations in the claim. Modifications of the disclosed embodiments incorporating the spirit and substance of the invention may occur to persons skilled in the art and such modifications are within the scope of the present invention. Furthermore, all references cited herein are incorporated by reference in their entirety.

We claim:

1. A medical composition comprising:
a light emitting composite adapted for inserting into a mammalian body, the light emitting composite comprising a matrix material comprising a light emitting agent, wherein the light emitting composite emits light of a wavelength range that is substantially non-absorbent to animal fluid or tissue.

2. The medical composition of claim 1, wherein the medical composition is a medical device.

3. The medical composition of claim 2, wherein the medicate composite is an implantable medical device.

4. The medical composition of claim 3, wherein the medical device is selected from the group consisting of a stent, a shunt, a filter, a graft, a lead, a scaffold, a plug, a mechanical heart valve, or another type of implant.

5. The medical composition of claim 2, wherein the medical device is a catheter.

6. The medical composition of claim 1, wherein the medical composition is a medical instrument.

7. The medical composition of claim 1, wherein the medical composition a pharmaceutical composition.

8. The medical composition of claim 7, wherein the pharmaceutical composition is a pill, liquid, ointment, cream or vapor.

9. The medical composition of claim 1, wherein the medical device emits light of a wavelength range from about 600 nm to about 1100 nm.

10. The medical composition of claim 1, wherein the light emitting material is a semiconductor nanocrystal composition comprising a semiconductor nanocrystal core having an outer surface.

11. The medical composition of claim 10, wherein the semiconductor nanocrystal composition comprises:
a shell of a semiconductor material formed on the outer surface of the semiconductor nanocrystal core.

12. The medical composition of claim 10, wherein the semiconductor nanocrystal composition comprises:
a metal layer formed on the outer surface of the semiconductor nanocrystal core.

13. The medical composition of claim 12, wherein the semiconductor nanocrystal composition comprises:
a shell of a semiconductor material overcoating the metal layer.

14. The medical composition of claim 1, wherein the light emitting material is a fluorescent optical agent.

15. The medical composition of claim 1, wherein the light emitting material is an inorganic phosphor.

16. The medical composition of claim 1, wherein the matrix material is a polymer.

17. The medical composition of claim 16, wherein the polymer comprises a plastic, a glass, or a suitable combination thereof.

18. The medical composition of claim 1, wherein the composite is an ink, paint, a dye, or a suitable combination thereof.

19. The medical composition of claim 1, comprising:
a biocompatible material overcoating the matrix material.

20. A system, comprising:
the medical composition of claim 1;
an excitation source capable of exciting the composite; and
a detection device capable of detecting an emission from the excited composite.

21. The system of claim 20, wherein the excitation source is selected from the group consisting of a white light source in optical communication with the light emitting composite; a light emitting diode in optical communication with the light emitting composite; a laser in optical communication with the light emitting composite; and a chemiluminescent material in optical communication with the light emitting composite.

22. The system of claim 20, wherein the detection device is a camera or a night vision scope tunable to the wavelength of the emission

23. The system of claim 20, wherein the detection device is a night vision scope tunable to the wavelength of the emission.

24. A method, comprising:
providing the medical composition of claim 1;
exciting the composite by transmitting a signal from an excitation source to the composite; and
detecting an emission from the excited composite.

25. The method of claim 24, wherein the transmitted signal is a beam of light.

26. The method of claim 24, wherein the emission is light of the wavelength range between about 600 nm and about 1100 nm.

27. The method of claim 24, further comprising:
generating an audible signal upon detecting the emission.

28. The method of claim 24, further comprising:
upon detecting the emission, triggering a second detection at the emission detection location using an alternate detecting agent.

29. The method of claim 28, wherein the alternate detecting agent is a contrast material detectable by x-ray imaging, positron emission tomography (PET), magnetic resonance imaging (MRI), or a suitable combination thereof.
